(19) United States
(12) Patent Application Publication
(10) Pub. No.: US 2015/0072935 A1
(43) Pub. Date: Mar. 12, 2015

(54) COMPOSITION AND EXTERNAL APPLICATION FOR ACCELERATION OF MUSCLE DIFFERENTIATION AND IMPROVING OF MUSCLE MASS

(71) Applicant: NEOPHARM CO., LTD., Daejeon (KR)

(72) Inventors: Bong-Woo KIM, Daejeon (KR); Jeong Eun JEON, Daejeon (KR); Se Kyoo JEONG, Daejeon (KR); Yoon KIM, Daejeon (KR)

(73) Assignee: NEOPHARM CO., LTD., Daejeon (KR)

(21) Appl. No.: 14/470,025

(22) Filed: Nov. 5, 2014

(30) Foreign Application Priority Data
Aug. 27, 2013 (KR) 10-2013-0101647

(51) Int. Cl. C07K 7/06

(52) U.S. Cl. CPC C07K 7/06
USPC 514/18.2; 514/21.8; 514/17.7

(57) ABSTRACT

Provided are a composition and an external application for promoting muscle differentiation or improving muscle mass containing an amino acid derivative promoting muscle cell differentiation, a composition and an external application for alleviating muscle function deterioration caused by muscle damage and recovering damaged muscle containing the same, or a pharmaceutical composition and an external application for treating or improving a muscle deterioration disease containing the same.

### CROSS-REFERENCE TO RELATED APPLICATIONS

This application claims priority under 35 U.S.C. §119 to Korean Patent Application No. 10-2013-0101647, filed on Aug. 27, 2013, in the Korean Intellectual Property Office, the disclosure of which is incorporated herein by reference in its entirety.

### TECHNICAL FIELD

The following disclosure relates to a composition and an external application for promoting muscle cell differentiation, recovering damaged muscles, and increasing muscle mass, and more particularly, to a composition and an external application for promoting muscle cell differentiation capable of developing muscles by promoting muscle cell differentiation or for recovering muscles damaged by exercise and increasing muscle mass.

In addition, the following disclosure relates to an external application and a pharmaceutical composition for treating and improving a muscle deterioration diseases.

### BACKGROUND

Muscles of our body are attached to bones to perform various functions. For example, the muscles serve to protect the bones, properly maintain a body shape, and the like.

Excessive exercise causes muscle fatigue and muscle damage and deteriorates exercise capacity. Muscle damage includes contusions (bruises), lacerations, ischemia, strains, and severe damage to skeletal muscles. The damage as described above may cause tremendous pain and can incapacitate the affected person by preventing them from being able to go to exercise and work or even to participate in normal daily activities. In the case of severe damage to the skeletal muscles, strain is most common. Muscle strain damage is characterized by a disruption of a muscle-tendon unit. The disruption of the muscle-tendon unit may occur anywhere on the muscle. Strains accounts for at least 30% of all damage treated by occupational therapy or sports medicine professionals.

Treatment capable of decreasing muscle damage or accelerating recovery of muscle tissues may accelerate recovery of muscle strength after exercise. This treatment may assist in recovery of the muscles after diseases.

The muscles promote calcium influx to increase a bone density. However, redistribution of body fats and body proteins in a human body occur as a change in constituent components in accordance with aging, and by the age of about 50, a synthesis rate of protein in muscle cells is decreased than a decomposition rate thereof, such that muscle degeneration rapidly starts, and a person may be exposed to a muscle deterioration disease.

Sarcopenia, which is one of the muscle deterioration diseases, means a state in which a usual body weight is decreased by about 13 to 24%, and in the case of sarcopenia patient, a behavior amount may be significantly decreased, which is harmful to mental health, life satisfaction may also be decreased, and the sarcopenia patient may be easily injured even in easy daily life to thereby be severely wounded.

As a cause of sarcopenia, there are a gradual decrease in amounts and quality of skeletal muscles caused by aging, a decrease in a weight containing fats and body fat components caused by improper dietary energy intake, and the like. Usually, sarcopenia is due to aging and is deeply associated with ages.

However, recently, as an interest in appearance and diet has been increased, sarcopenia may occur due to a rapid decrease in a body weight regardless of ages, and researches and efforts for treating muscle deterioration caused by general muscle deterioration decreases or increasing muscle mass have been intensively conducted.

As an example, uses of Ginkgo biloba extracts for preparing a medicine for treating sarcopenia has been disclosed in Korean Patent Publication No. 10-0891393, but research into a technology for treating the muscle deterioration disease and improving the muscle mass has been still required.

### SUMMARY

An embodiment of the present invention is directed to providing a composition and an external application for promoting muscle cell differentiation and increasing muscle mass containing an amino acid derivative promoting muscle cell differentiation or a salt thereof.

Another embodiment of the present invention is directed to providing a composition for alleviating muscle function deterioration caused by muscle damage and enhancing recovery from the muscle damage, more particularly, to providing a composition and an external application for alleviating muscle function deterioration caused by muscle damage and enhancing recovery from the muscle damage containing an amino acid derivative promoting muscle cell differentiation or a pharmaceutically acceptable salt thereof as an active component.

Another embodiment of the present invention is directed to providing a pharmaceutical composition and an external application for alleviating muscle function deterioration caused by muscle damage and enhancing recovery from the muscle damage containing an amino acid derivative promoting muscle cell differentiation or a pharmaceutically acceptable salt thereof as an active component.

Another embodiment of the present invention is directed to providing a composition and an external application for treating or improving a muscle deterioration disease, and more particularly, to providing a pharmaceutical composition and an external application for treating or improving a muscle deterioration disease by using an amino acid derivative promoting muscle cell differentiation or a pharmaceutically acceptable salt thereof as an active component.

In one general aspect, a composition and an external application for promoting muscle cell differentiation or increasing muscle mass by using an amino acid derivative represented by the following Chemical Formula 1 or a salt thereof.

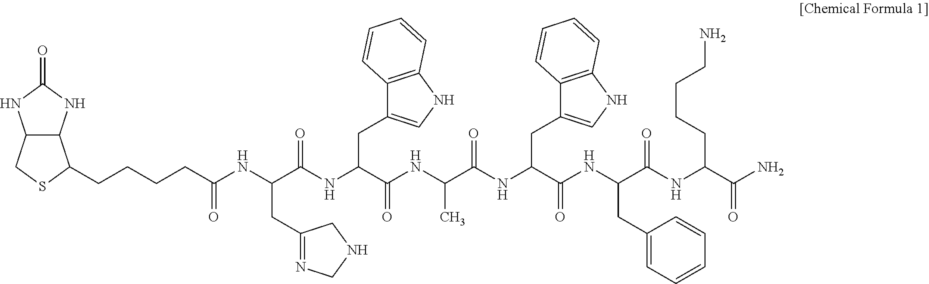 [SMILES: CC(NC(=O)C(Cc1c[nH]c2ccccc12)NC(=O)C(CC1=NCNC1)NC(=O)CCCCC1SCC2NC(=O)NC21)C(=O)NC(Cc1c[nH]c2ccccc12)C(=O)NC(Cc1ccccc1)C(=O)NC(CCCCN)C(N)=O]

The composition for promoting muscle cell differentiation or increasing muscle mass according to an exemplary embodiment of the present invention may contain the amino acid derivative represented by Chemical Formula 1 or the salt thereof at a content of 0.001 to 1 wt %.

In another general aspect, a composition and an external application for alleviating muscle function deterioration and enhancing recovery from muscle damage contain an amino acid derivative represented by Chemical Formula 1 or a salt thereof.

The composition and the external application for alleviating muscle function deterioration and enhancing recovery from muscle damage according to an exemplary embodiment of the present invention may contain the amino acid derivative represented by Chemical Formula 1 or the salt thereof at a content of 0.001 to 1 wt %.

In another general aspect, a pharmaceutical composition and an external application for alleviating muscle function deterioration and enhancing recovery from muscle damage contain an amino acid derivative represented by Chemical Formula 1 or a salt thereof at a content of 0.001 to 1 wt %.

In another general aspect, a pharmaceutical composition for treating or improving a muscle deterioration disease contains an amino acid derivative represented by Chemical Formula 1 or a pharmaceutically acceptable salt thereof as an active component.

The pharmaceutical composition for treating or improving a muscle deterioration disease according to an exemplary embodiment of the present invention may contain the amino acid derivative represented by Chemical Formula 1 or the pharmaceutically acceptable salt thereof at a content of 0.001 to 1 wt %.

The pharmaceutical composition according to an exemplary embodiment of the present invention may be used in a form of tablets, pills, capsules, granules, powders, solutions, patches, or injections.

The pharmaceutical composition according to an exemplary embodiment of the present invention may promote muscle cell differentiation, such that recovery from muscle damage may be improved.

In addition, the pharmaceutical composition according to an exemplary embodiment of the present invention may promote muscle cell differentiation, such that the muscle deterioration disease may be treated or improved.

According to an exemplary embodiment of the present invention, the muscle deterioration disease may be one or more selected from the group consisting of sarcopenia, muscular dystrophy, rigid spinesyndrome, muscle-eye-brain disease, amyotrophic lateral sclerosis (Lou Gehrig's disease), Charcot-Marie-Tooth disease, chronic inflammatory neuropathy, and distal myopathy.

In another general aspect, an external application for alleviating muscle function deterioration and enhancing recovery from muscle damage contains an amino acid derivative represented by Chemical Formula 1 or a salt thereof.

The external application for alleviating muscle function deterioration and enhancing recovery from muscle damage according to an exemplary embodiment of the present invention may contain the amino acid derivative represented by Chemical Formula 1 or the salt thereof at a content of 0.001 to 1 wt %.

In another general aspect, an external application for treating or improving a muscle deterioration disease contains an amino acid derivative represented by Chemical Formula 1 or a pharmaceutically acceptable salt thereof as an active component.

The external application for treating or improving a muscle deterioration disease according to an exemplary embodiment of the present invention may contain the amino acid derivative represented by Chemical Formula 1 or the pharmaceutically acceptable salt thereof at a content of 0.001 to 1 wt %.

Other features and aspects will be apparent from the following detailed description, the drawings, and the claims.

### BRIEF DESCRIPTION OF THE DRAWINGS

FIG. 1 is a graph showing a result of muscle cell differentiation in Example 1 according to the present invention.

FIG. 2 is a graph showing a result of muscle differentiation promotion depending on differentiation time in Example 2 according to the present invention.

### DETAILED DESCRIPTION OF EMBODIMENTS

The present invention provides a composition and an external application for promoting muscle differentiation or improving muscle mass containing an amino acid derivative represented by the following Chemical Formula 1 or a salt thereof.

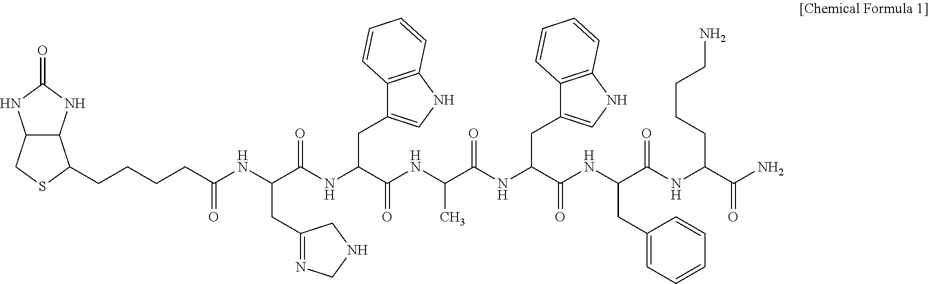 [SMILES: CC(NC(=O)C(Cc1c[nH]c2ccccc12)NC(=O)C(CC1=NCNC1)NC(=O)CCCCC1SCC2NC(=O)NC21)C(=O)NC(Cc1c[nH]c2ccccc12)C(=O)NC(Cc1ccccc1)C(=O)NC(CCCCN)C(N)=O]

The composition and the external application according to the present invention contain the amino acid derivative represented by Chemical Formula 1 or the salt thereof to promote muscle cell differentiation and improve muscle mass, such that the composition for promoting muscle differentiation or improving muscle mass according to the present invention may be usefully used to treat diseases or used for pharmaceutical or cosmetic purposes by promoting muscle differentiation and improving muscle mass.

In the composition and an external application for promoting muscle differentiation or improving muscle mass according to the present invention, as the salt of the amino acid derivative represented by Chemical Formula 1, any salt may be used as long as it is a chemically acceptable salt, but a pharmaceutically acceptable salt may be preferable. The pharmaceutically acceptable salt will be described below.

In addition, the present invention provides a composition and an external application for alleviating muscle function deterioration and recovering muscle functions and muscles from muscle damage, and the composition and the external application as described above contain an amino acid derivative represented by Chemical Formula 1.

The present invention provides a muscle mass improving composition, a pharmaceutical composition, and an external application for promoting development of the muscles and treating and improving a muscle deterioration disease. The pharmaceutical composition and the application for treating and improving a muscle deterioration disease contain an amino acid derivative represented by Chemical Formula 1 or a pharmaceutically acceptable salt thereof as an active component.

In the amino acid derivative presented by Chemical Formula 1, hexapeptide-2 (GHRP-2) and biotin are bonded to each other, and the amino acid derivative presented by Chemical Formula 1 may contain one or more asymmetric carbon atoms and exist in racemic and optically active form. All of the compounds as described above and the enantiomers thereof are included in the scope of the present invention.

More preferably, the amino acid derivative represented by Chemical Formula 1 may be represented by the following Chemical Formula 2.

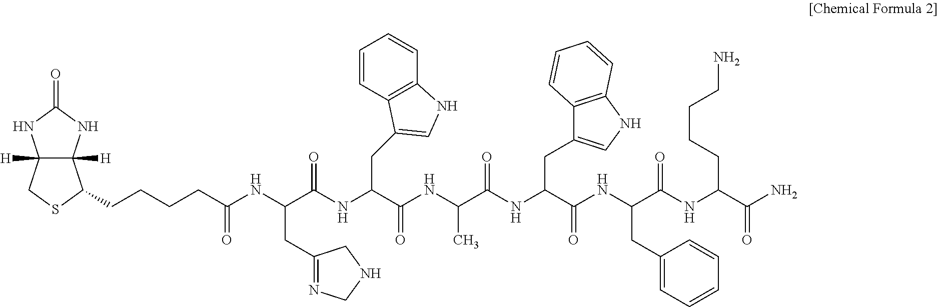 [SMILES: CC(NC(=O)C(Cc1c[nH]c2ccccc12)NC(=O)C(CC1=NCNC1)NC(=O)CCCC[C@@H]1SC[C@@H]2NC(=O)N[C@@H]21)C(=O)NC(Cc1c[nH]c2ccccc12)C(=O)NC(Cc1ccccc1)C(=O)NC(CCCCN)C(N)=O]

The amino acid derivative represented by Chemical Formula 1 according to the present invention has a significant effect on muscle differentiation, such that the amino acid derivative is significantly useful to promote muscle differentiation or improve muscle mass. In addition, the amino acid derivative is significantly useful to recovery the damaged muscles and treat and improve the muscle deterioration disease.

In addition, the pharmaceutical composition and the external application for treating and improving a muscle deterioration disease according to the present invention contain the amino acid derivative represented by Chemical Formula 1 promoting muscle differentiation, such that the pharmaceutical composition and the external application have a significant effect on regeneration, growth, and elasticity improvement of muscle cells due to muscle cell differentiation promotion.

The pharmaceutical composition and the external application for promoting muscle differentiation, recovering damaged muscles, or treating or improving a muscle deterioration disease according to the present invention contain the amino acid derivative represented by Chemical Formula 1 or the pharmaceutically acceptable salt thereof at a content of 0.001 to 1 wt %. In view of effects and economical aspects, more preferably, the content may be 0.005 to 0.1 wt %.

A preferable dose of the pharmaceutical composition according to the present invention may be variously prescribed depending on factors such as a formulation method, an administration method, an age, a weight, sex, a disease state of a patient, foods, an administration time, an administration route, an excretion rate, and response sensitivity. For example, a daily dose of the pharmaceutical composition according to the present invention may be 0.001 to 100 mg/kg.

The pharmaceutical composition according to the present invention may be orally or parenterally administered, and in the case of parenteral administration, the pharmaceutical composition may be intravenously, subcutaneously, intramuscularly, and intraperitoneally injected, or transdermally administered, or the like.

The pharmaceutical composition according to the present invention may contain a pharmaceutically acceptable carrier. The pharmaceutically acceptable carrier contained in the pharmaceutical composition according to the present invention, which is generally used at the time of preparation, includes lactose, dextrose, sucrose, sorbitol, mannitol, starches, acacia rubber, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, and the like, but is not limited thereto. The pharmaceutical composition according to the present invention may additionally include a lubricant, a wetting agent, a sweetener, a flavoring agent, an emulsifier, a suspending agent, a preservative, or the like, in addition to the above components.

The pharmaceutical composition according to the present invention may be prepared in a unit dose form or prepared in a form in which the pharmaceutical composition is input into a multi-dose container by being formulated in a general formulation using the pharmaceutically acceptable carrier and/or the excipient according to a method capable of being performed by those skilled in the art to which the present invention pertains. The general formulation means, for example, oral administration formulations (tablets, capsules, powders) and formulations for sublingual, rectal, intravaginal, nasal, topical or parenteral administration (including intravenous, intracavernosal, intramuscular, subcutaneous, and intratubular administration). For example, the compound according to the present invention may be orally or sublingually administered in a form of tablets containing starch or lactose, capsules containing only the compound according to the present invention or containing an excipient in addition to the compound, or elixirs or suspensions containing a chemical for flavor or color. A liquid formulation is prepared together with a pharmaceutically acceptable additive such as a suspending agent (for example, methylcellulose, semi-synthetic glycerides such as Witepsol, glyceride mixture such as a mixture of apricot kernel oil and polyethylene glycol (PEG)-6 ester or a mixture of PEG-8 and caprylic/capric glyceride). Further, in the case in which the pharmaceutical composition is parenterally, for example, intravenously, intracavernosally, intramuscularly, subcutaneously, and intratubularly injected, it is most preferable that the pharmaceutical composition is used in a form of a sterile aqueous solution. In this case, the solution may contain other materials (for examples, salts or monosaccharide such as mannitol or glucose) so as to be isotonic with blood.

More specifically, the pharmaceutical composition according to an exemplary embodiment of the present invention may be used in a form of tablets, pills, capsules, granules, powders, solutions, patches, transdermal absorption patches using a microneedle, or injections.

The amino acid derivative represented by Chemical Formula 1 according to the present invention may be used in a form of the pharmaceutically acceptable salt, and as the salt, an acid addition salt formed by a pharmaceutically acceptable free acid may be used. The acid addition salt is obtained from inorganic acids such as hydrochloric acid, nitric acid, phosphoric acid, sulfuric acid, hydrobromic acid, hydriodic acid, nitrous acid, or phosphorous acid, non-toxic organic acids such as aliphatic mono- and di-carboxylate, phenyl-substituted alkanoates, hydroxy alkanoates, and alkanedioates, aromatic acids, aliphatic and aromatic sulfonic acids, and organic acids such as acetic acid, benzoic acid, citric acid, lactic acid, maleic acid, gluconic acid, methanesulfonic acid, 4-toluenesulfonic acid, tartaric acid, fumaric acid. Pharmaceutically non-toxic salts as described above include sulfate, pyrosulfate, bisulfate, sulfide, bisulfide, nitrate, phosphate, monohydrogen phosphate, dihydrogen phosphate, methaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dionate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzene sulfonate, xylenesulfonate, phenylacetate, phenylpropionate, phenylbutyrate, citrate, lactate, β-hydroxybutyrate, glycolate, tartrate, methanesulfonate, propanesulfonate, naphthalene-1-sulfonate, naphthalene-2-sulfonate, or mandelate, but are not limited thereto.

The acid addition salt according to the present invention may be prepared by a general method. For example, the acid addition salt may be prepared by dissolving the amino acid derivative of Chemical Formula 1 in an organic solvent, for example, methanol, ethanol, acetone, methylene chloride, acetonitrile, or the like, adding an organic or inorganic acid, and filtering and drying the produced precipitates, or distilling a solvent and an excess acid under reduced pressure, followed by drying or crystallization in the presence of an organic solvent.

In addition, a pharmaceutically acceptable metal salt may be prepared using a base. An alkaline metal or alkaline earth metal salt may be prepared, for example, by dissolving the compound in an excess alkaline metal hydroxide or alkaline earth metal hydroxide solution, filtering non-soluble compound salt, and evaporating and drying the filtrate. In this case, as the metal salt, it is pharmaceutically suitable to prepare a sodium, potassium, or calcium salt. In addition, a silver salt corresponding thereto may be obtained by reacting the alkaline metal or alkaline earth metal salt with a suitable silver salt (for example, silver nitrate). Further, the present invention includes all of the solvates, hydrates, stereoisomers, and the like, capable of being prepared from therefrom as well as the amino acid derivative represented by Chemical Formula 1 and the pharmaceutically acceptable salt thereof.

In addition, the amino acid derivative of Chemical Formula 1 according to the present invention may exist in a solvate form as well as a non-solvate form in a pharmaceutically acceptable solvent, for example, water, ethanol, or the like. Generally, for objects of the present invention, it is considered that the solvate form is equivalent to the non-solvate form. The amino acid derivative of Chemical Formula 1 may exist in one or more crystalline states. That is, the amino acid derivative may exist as a cocrystal, a polymorph, or an amorphous solid. All of the above-mentioned forms are included in the present invention and claims of the present invention.

In the pharmaceutical composition according to an exemplary embodiment of the present invention, the amino acid derivative represented by Chemical Formula 1 promotes muscle cell differentiation, such that the pharmaceutical composition may be very usefully used to recover and improve muscles damaged due to exercise or work and treat or improve the muscle deterioration disease

According to an exemplary embodiment of the present invention, the muscle deterioration disease may be one or more selected from the group consisting of sarcopenia, muscular dystrophy, rigid spinesyndrome, muscle-eye-brain disease, amyotrophic lateral sclerosis (Lou Gehrig's disease), Charcot-Marie-Tooth disease, chronic inflammatory neuropathy, and distal myopathy.

Further, the present invention provides the external application for treating or improving a muscle deterioration disease containing an amino acid derivative represented by Chemical Formula 1 or a pharmaceutically acceptable salt thereof. This external application may contain the amino acid derivative represented by Chemical Formula 1 or the pharmaceutically acceptable salt thereof at a content of 0.001 to 1 wt %, preferably 0.005 to 0.1 wt %.

Hereinafter, the present invention will be described in detail through Examples. However, the following Examples are to illustrate the present invention, and the scope of the present invention is not limited to the following Examples.

### Example 1

### Muscle Differentiation Promotion by Composition According to Present Invention

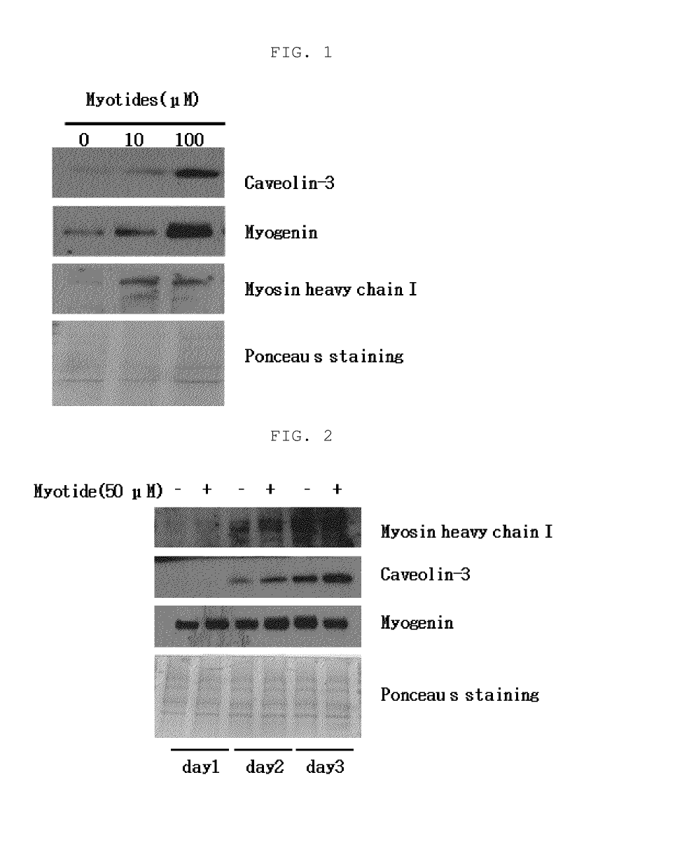

Undifferentiated C2C12 myoblast cells were cultured in a dish having a diameter of 100 mm using a Dulbecco's modified Eagle's medium (DMEM) with 10% fetal bovine serum (FBS). When the cells covered about 70% of an area, the cells were seeded in a 6-well dish at 8×10⁵/well and cultured until the cells covered 90% of an area. Thereafter, muscle differentiation was induced using a differentiation medium with 2% horse serum. In this case, the cells were treated with a myotide (Chemical Formula 2, Incospharm Corp.) composition (0.1% DMSO in phosphate buffer saline) of the present invention at contents of 0, 10, and 100 uM. The differentiation medium was changed into a new differentiation medium containing biotides every 24 hours, and after 3 days of differentiation induction, muscles cell lysis was performed using a cell lysis buffer, followed by western blotting. In order to confirm a degree of muscle differentiation, protein expression amounts of myosin heavy chain I, caveolin-3, and myogenin, which are representative protein of muscles, were observed. As shown in FIG. 1, it was confirmed that in the case of inducing muscle differentiation by treating the composition according to the present invention, expression of muscle proteins was concentration-dependently increased. Therefore, the composition according to the present invention promoted muscle differentiation.

### Example 2

### Muscle Differentiation Promotion by Composition According to Present Invention Depending on Differentiation Time

Undifferentiated C2C12 myoblast cells were cultured in a dish having a diameter of 100 mm using a DMEM with 10% fetal bovine serum (FBS). When the cells covered about 70% of an area, the cells were seeded in a 6-well dish at 8×10⁵/well and cultured until the cells covered 90% of an area. Thereafter, muscle differentiation was induced using a differentiation medium with 2% horse serum. In this case, the cells were treated with a myotide (Chemical Formula 2, Incospharm Corp.) composition (0.1% DMSO in phosphate buffer saline) of the present invention at a content of 50 uM. After 1, 2, and 3 days of differentiation induction, cell lysis was performed using a lysis buffer, and a degree of muscle differentiation was confirmed by western blotting. As shown in FIG. 2, it was confirmed that in the case of inducing muscle differentiation by treating the composition according to the present invention, expression of muscle proteins (myosin heavy chain I, caveolin-3, and myogenin) was increased.

Therefore, it may be appreciated that the amino acid derivative represented by Chemical Formula 1 according to the present invention may significantly promote muscle cell differentiation.

The composition for promoting muscle differentiation or improving muscle mass according to the present invention contains the amino acid derivative represented by Chemical Formula 1 promoting muscle cell differentiation or the salt thereof, such that the composition may be usefully used to promote muscle differentiation and improve muscle mass.

In addition, since the composition for alleviating muscle function deterioration and enhancing recovery from muscle damage according to the present invention contains the amino acid derivative represented by Chemical Formula 1 promoting muscle cell differentiation or the salt thereof, the muscle cell differentiation may be promoted, such that the composition may be significantly effective for recovering and improving damaged muscles.

In addition, since the pharmaceutical composition for alleviating muscle function deterioration and enhancing recovery from muscle damage according to the present invention contains the amino acid derivative represented by Chemical Formula 1 promoting muscle cell differentiation or the pharmaceutically acceptable salt thereof, the muscle cell differentiation may be promoted, such that the pharmaceutical composition may be significantly effective for recovering and improving damaged muscles.

Further, since the composition for treating or improving a muscle deterioration disease according to the present invention contains the amino acid derivative represented by Chemical Formula 1 promoting muscle cell differentiation or the pharmaceutically acceptable salt thereof, the muscle cell differentiation may be promoted, such that the composition may be significantly effective for treating or improving the muscle deterioration disease.

In addition, since the external application for treating or improving a muscle deterioration disease according to the present invention contains the amino acid derivative represented by Chemical Formula 1 or the pharmaceutically acceptable salt thereof, the external application is significantly effective for improving growth of muscles and increasing elasticity of the muscles in addition to rapid recovery from muscle damage by injuries.

Further, the external application for promoting muscle differentiation or improving muscle mass according to the present invention containing the amino acid derivative represented by Chemical Formula 1 or the salt thereof may promote muscle differentiation to assist in growth of the muscles and increase elasticity of the muscles, such that the external application may be usefully used as an external application of improving muscle mass for beauty.

1. A composition for promoting muscle cell differentiation or increasing-muscle mass comprising an amino acid derivative represented by the following Chemical Formula 1 or a salt thereof:

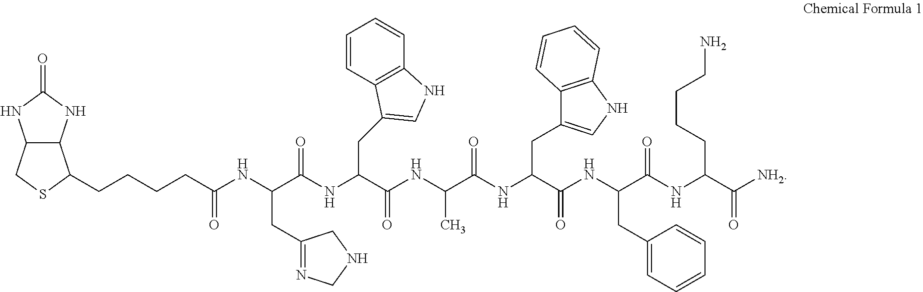 [SMILES: CC(NC(=O)C(Cc1c[nH]c2ccccc12)NC(=O)C(CC1=NCNC1)NC(=O)CCCCC1SCC2NC(=O)NC21)C(=O)NC(Cc1c[nH]c2ccccc12)C(=O)NC(Cc1ccccc1)C(=O)NC(CCCCN)C(N)=O]

2. The composition for promoting muscle cell differentiation or increasing muscle mass of claim 1, wherein the amino acid derivative represented by Chemical Formula 1 or the salt thereof is contained at a content of 0.001 to 1 wt %.

3. A composition for alleviating muscle function deterioration caused by muscle damage and enhancing recovery from the muscle damage comprising an amino acid derivative represented by the following Chemical Formula 1 or a salt thereof:

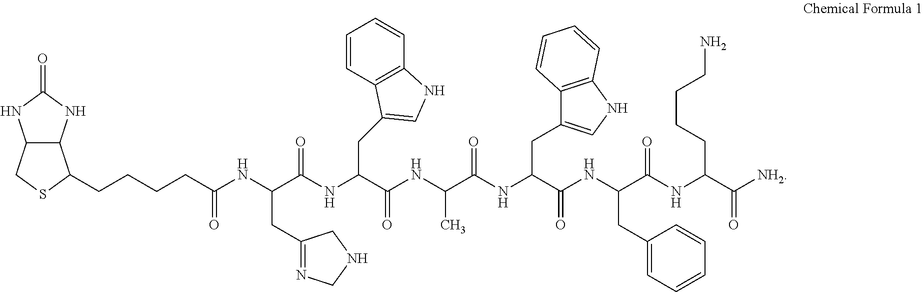 [SMILES: CC(NC(=O)C(Cc1c[nH]c2ccccc12)NC(=O)C(CC1=NCNC1)NC(=O)CCCCC1SCC2NC(=O)NC21)C(=O)NC(Cc1c[nH]c2ccccc12)C(=O)NC(Cc1ccccc1)C(=O)NC(CCCCN)C(N)=O]

4. The composition for alleviating muscle function deterioration caused by muscle damage and enhancing recovery from the muscle damage of claim 3, wherein the amino acid derivative represented by Chemical Formula 1 or the salt thereof is contained at a content of 0.001 to 1 wt %.

5. An external application for promoting muscle cell differentiation or increasing muscle mass comprising an amino acid derivative represented by the following Chemical Formula 1 or a salt thereof:

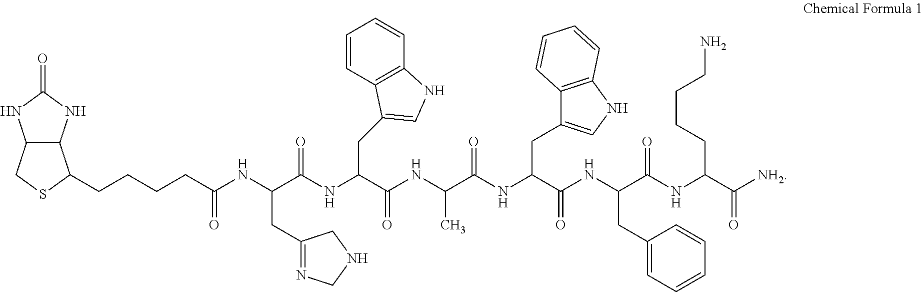 [SMILES: CC(NC(=O)C(Cc1c[nH]c2ccccc12)NC(=O)C(CC1=NCNC1)NC(=O)CCCCC1SCC2NC(=O)NC21)C(=O)NC(Cc1c[nH]c2ccccc12)C(=O)NC(Cc1ccccc1)C(=O)NC(CCCCN)C(N)=O]

6. The external application for promoting cell muscle differentiation or increasing muscle mass of claim 5, wherein the amino acid derivative represented by Chemical Formula 1 or the salt thereof is contained at a content of 0.001 to 1 wt %.

7. An external application for alleviating muscle function deterioration caused by muscle damage and recovering from the muscle damage comprising an amino acid derivative represented by the following Chemical Formula 1 or a pharmaceutically acceptable salt thereof as an active component:

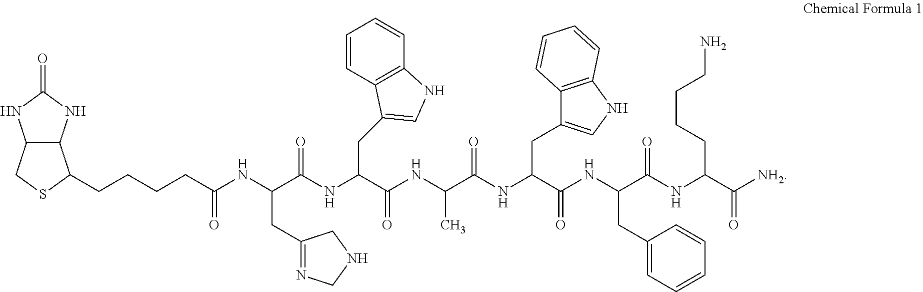 [SMILES: CC(NC(=O)C(Cc1c[nH]c2ccccc12)NC(=O)C(CC1=NCNC1)NC(=O)CCCCC1SCC2NC(=O)NC21)C(=O)NC(Cc1c[nH]c2ccccc12)C(=O)NC(Cc1ccccc1)C(=O)NC(CCCCN)C(N)=O]

8. The external application for alleviating muscle function deterioration caused by muscle damage and recovering from the muscle damage of claim 7, wherein the amino acid derivative represented by Chemical Formula 1 or the pharmaceutically acceptable salt thereof is contained at a content of 0.001 to 1 wt %.

9. A pharmaceutical composition for treating or improving a muscle deterioration disease comprising an amino acid derivative represented by the following Chemical Formula 1 or a pharmaceutically acceptable salt thereof as an active component:

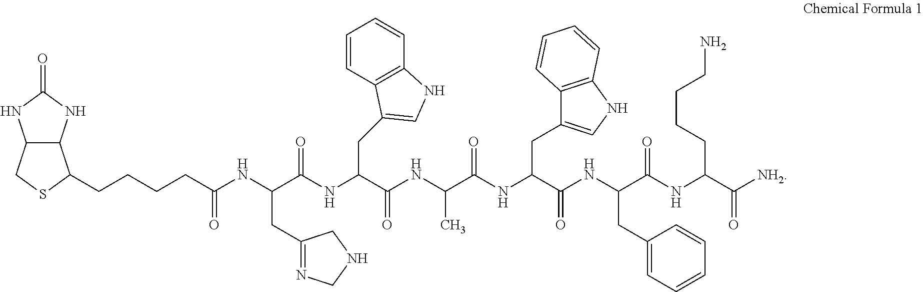 [SMILES: CC(NC(=O)C(Cc1c[nH]c2ccccc12)NC(=O)C(CC1=NCNC1)NC(=O)CCCCC1SCC2NC(=O)NC21)C(=O)NC(Cc1c[nH]c2ccccc12)C(=O)NC(Cc1ccccc1)C(=O)NC(CCCCN)C(N)=O]

10. The pharmaceutical composition for treating or improving a muscle deterioration disease of claim 9, wherein the amino acid derivative represented by Chemical Formula 1 or the pharmaceutically acceptable salt thereof is contained at a content of 0.001 to 1 wt %.

11. The pharmaceutical composition for treating or improving a muscle deterioration disease of claim 9, wherein it is used in a form of tablets, pills, capsules, granules, powders, solutions, patches, or injections.

12. The pharmaceutical composition for treating or improving a muscle deterioration disease of claim 9, wherein the muscle deterioration disease is treated or improved by muscle cell differentiation promotion.

13. The pharmaceutical composition for treating or improving a muscle deterioration disease of claim 9, wherein the muscle deterioration disease is one or more selected from the group consisting of sarcopenia, muscular dystrophy, rigid spinesyndrome, muscle-eye-brain disease, amyotrophic lateral sclerosis (Lou Gehrig's disease), Charcot-Marie-Tooth disease, chronic inflammatory neuropathy, and distal myopathy.

14. An external application for treating or improving a muscle deterioration disease comprising an amino acid derivative represented by the following Chemical Formula 1 or a pharmaceutically acceptable salt thereof as an active component:

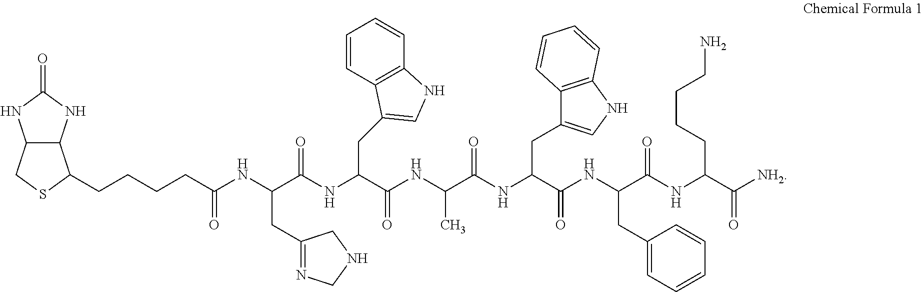 [SMILES: CC(NC(=O)C(Cc1c[nH]c2ccccc12)NC(=O)C(CC1=NCNC1)NC(=O)CCCCC1SCC2NC(=O)NC21)C(=O)NC(Cc1c[nH]c2ccccc12)C(=O)NC(Cc1ccccc1)C(=O)NC(CCCCN)C(N)=O]

15. The external application for treating or improving a muscle deterioration disease of claim 14, wherein the amino acid derivative represented by Chemical Formula 1 or the pharmaceutically acceptable salt thereof is contained at a content of 0.001 to 1 wt %.
